(19) United States
(12) Patent Application Publication
(10) Pub. No.: US 2011/0256236 A1
(43) Pub. Date: Oct. 20, 2011

(54) Enhancement of the Anti-Leukemic Effect of Thalidomide

(76) Inventors: Erian Girgis, Tallahassee, FL (US); Magdi Soliman, Tallahassee, FL (US)

(21) Appl. No.: 12/761,500

(22) Filed: Apr. 16, 2010

(51) Int. Cl. A61K 33/36; A61P 35/00; A61P 35/02; A61K 31/454

(52) U.S. Cl. 424/623; 514/323

(57) ABSTRACT

The cytotoxic effects of thalidomide are enhanced when it is used in combination with other chemotherapeutic agents, particularly arsenic trioxide, indicating benefits for the treatment of blood-related cancers, especially acute myelogenous leukemia.

### STATEMENT REGARDING FEDERALLY SPONSORED RESEARCH OR DEVELOPMENT

This research was supported in part by grant number NIH/RCMI RR03020 from the National Institutes of Health.

### FIELD OF THE INVENTION

The present invention relates generally to the field of anti-cancer therapy. More particularly, it provides compositions and methods for using thalidomide in combination with another chemotherapeutic agent such as an arsenical compound for treating cancers such as leukemia, more specifically acute myelogenous leukemia (AML).

### BACKGROUND OF THE INVENTION

Acute myeloid leukemia (AML) is a heterogeneous malignant disease with diverse biological features. Approximately 60% of AML cases are in patients older than 60 years (Godwin, J. E. and Smith S. E. Acute Myeloid Leukemia in the Older Patient, Crit. Rev. Oncol. Hematol. 2003; 48S: pp. S17-S26). These elderly patients usually do not respond as well to conventional chemotherapy as younger AML patients. This is due to the intrinsic resistant nature of their leukemic cells and/or their poor tolerance to conventional chemotherapy regimens (Harousseuau, J. L., Acute Myeloid Leukemia in the Elderly. Blood Rev. 1988; 12: 145.153). Very little progress has been made over the past four decades in the treatment outcome of AML especially in elderly patients. In general only 25% to 30% of adult patients are cured (Cortes, J. E., et al., Acute Lymphocytic Leukemia: A Comprehensive Review with emphasis on Biology and Therapy, Cancer, 1995; 76:2393-2417; Estey, E. H., et al., Therapy for Acute Myeloid Leukemia, in Hoffman R, Benz E Jr, et, al, editors, Hematology: Basic Principles and Practice, 2^{nd} ed. New York, N.Y., Churchill Livingstone, 1994; pp 1014-1028; and Thomas, D. A. et al., Primary Refractory and Relapsed Adult acute Lymphoblastic Leukemia: Characteristics, Treatment Results, and Prognosis With Salvage Therapy, Cancer, 1999; 86: 1216-1230). Although prognosis varies among AML subtypes, most patients relapse following an initial complete response (CR) and ultimately die of resistant disease. Patients with AML who experience a particularly short first CR and those who fail to achieve CR after two induction attempts are unlikely to respond to any currently available chemotherapeutic agents. Similarly, patients with high-risk myelodysplastic syndromes (MDS) likely to progress to AML (refractory anemia with excess blasts [RAEB] or refractory anemia with excess blasts in transformation [RAEBT]) have an estimated survival of less than one year (Berm M., et al., Topotecan and Cytarabine is An Active Combination Regimen in Myelodysplastic Syndromes and Chronic Myelomonocytic Leukemia, J Clin Oncol. 1999; 17: 2819-2830). The standard of care for this population remains supportive therapy since intensive chemotherapy regimens, such as used in AML, have been reported to produce high rates of treatment-related modality with rare durable remissions (Estey E. H., et al., High Remission Rate, Short Remission Duration In Patients With Refractory Anemia With Excess Blasts (RAEB) In Transformation (RAEB-t) Given Acute Myelogenous Leukemia (AML)-type Chemotherapy in Combination With Granulocyte-CSF (G-CSF), Cytokines Mol. Ther., 1995; 1: 21-28; and Thomas, D., Pilot Studies of Thalidomide In Acute Myelogenous Leukemia, Myelodysplastic Syndromes, and Myeloproliferative Disorders, Seminars in Oncology, 2000; 37 (1, Suppl 3): 26-34).

Therefore novel approaches and alternative therapeutic strategies need to be explored. It is apparent that what is needed is an alternative therapy for treating patients having AML.

The role of angiogenesis in hematologic malignancies has been elucidated by several investigators; hence, inhibitors of angiogenesis are being studied in these disorders. Thalidomide (Thalomid®, α-(N-phthalimido) glutarimide has both antiangiogenic and immunomodulatory properties. The drug inhibits angiogenesis by blocking basic fibroblast growth factor (bFGF) and vascular endothelial growth factor (VEGF), modulates various cytokines, enhances cell-mediated immunity by directly co-stimulating T cells, and alters adhesion molecule expression. However, thalidomide has shown only modest activity as a single agent in the therapy of AML. In addition, there is no study in the literature that identifies a dose or schedule that should be followed when using thalidomide.

We have discovered that the antileukemic effect of thalidomide can be increased when thalidomide is used in the right combination with another chemotherapeutic agent like arsenic trioxide. We believe that the antileukemic effect of thalidomide can also be increased when combined with IL-2 (Interleukin-2).

We have tested the effect of the combination of thalidomide and other chemotherapeutic agents on the KG-1a cell line (human acute myelogenous leukemia with early phenotype). The present study was conducted to test the hypothesis that combining thalidomide with certain chemotherapeutic agents will be a more effective therapeutic agent in the treatment and/or prevention of AML and other cancers.

### SUMMARY OF THE INVENTION

The present invention is based upon the discovery that the chemotherapeutic agent thalidomide has a synergistic effect with certain other chemotherapeutic agents, such as arsenical compounds, when used together in the treatment of cancer.

In accordance with the purpose(s) of this invention, as embodied and broadly described herein, this invention in one aspect relates to thalidomide and arsenical pharmaceutical compositions having anti-cancer activity. The compositions are useful as therapeutic agents for the treatment and/or prevention of acute myeloid leukemia.

In another aspect, the invention relates to a method of treating a patient with cancer comprising administering to the individual a therapeutically effective amount of a composition comprising thalidomide and an arsenical compound.

In another aspect the invention relates to administering a composition of thalidomide and an arsenical compound in combination with another agent or therapy.

Therapeutic formulations include thalidomide, an arsenical compound, and a pharmaceutically acceptable carrier.

### BRIEF DESCRIPTION OF THE DRAWINGS

FIG. 1 illustrates the early apoptosis of KG1-a cells incubated with various chemotherapeutic agents. Bars represent mean±SE. Cells were incubated for 48 hours with thalidomide (Th) (5 mg/ml), arsenic trioxide (As) (4 μM), or interleukin-2 (IL) (2001 U/ml), or combinations thereof.

FIG. 2 illustrates the late apoptosis of KG1-a cells incubated with various chemotherapeutic agents as in FIG. 5. The asterisk (*) indicates that it is significantly different from the control (p≦0.05). The double asterisk (**) indicates that it is significantly different from the control and the thalidomide treated cells (p≦0.05).

FIG. 3 illustrates the necrosis of KG1-a cells incubated with various chemotherapeutic agents as in FIG. 1.

### DETAILED DESCRIPTION OF THE INVENTION

The invention is described here in detail using the terms described below unless otherwise specified.

It must be noted that as used in the specification, and the appended claims, the singular forms “a,” “an” and “the” include plural referents unless the context clearly dictates otherwise.

Leukemia is a malignant cancer of the bone marrow and blood. It is characterized by the uncontrolled growth of blood cells. The common types of leukemias are divided into four categories: acute or chronic myelogenous, involving the myeloid elements of the bone marrow (white cells, red cells, megakaryocytes) and acute or chronic lymphocytic, involving the lymphoid lineage.

The term acute leukemia is meant to describe a rapidly progressing disease that results in the massive accumulation of immature, functionless cells (blasts) in the marrow and the blood. The marrow often can no longer produce enough normal red and white blood cells and platelets. Anemia, a deficiency of red cells, develops in virtually all leukemia patients. The lack of normal white cells impairs the body's ability to fight infections. A shortage of platelets results in bruising and easy bleeding. In general acute leukemia, unlike the chronic form, is potentially curable by elimination of the neoplastic clone.

Chronic leukemia progresses more slowly and leads to unregulated proliferation and hence marked over expression of a spectrum of mature (differentiated) cells.

A large majority of all cases of leukemia occur in persons over 60. The most common types of leukemia in adults are acute myelogenous leukemia (AML), chronic lymphocytic leukemia (CLL), and chronic myeloid leukemia (CML).

The term arsenical compound is meant to describe compounds such as arsenic trioxide (As₂O₃) and other arsenical salts.

The term “effective amount” means that amount of a drug or pharmaceutical agent that will elicit the biological or medical response of a tissue, cell, system, animal, or human that is being sought by a researcher or clinician. The term “therapeutically effective amount” means any amount which, as compared to a corresponding subject who has not received such amount, results in improved treatment, healing, prevention, or amelioration of a disease or disorder, or a decrease in the rate of advancement of a disease or disorder, and also includes amounts effective to enhance normal physiological function.

The terms “treatment of cancer” and “cancer treatment” refer to partially or totally inhibiting, delaying or preventing the progression of cancer including cancer metastasis; inhibiting, delaying, or preventing the recurrence of the cancer including cancer metastasis; or preventing the onset or development of cancer (chemoprevention) in a mammal, including humans.

The terms “patient” and “subject” as used herein refers to any vertebrate animal, preferably a mammal, and more preferably humans.

The phrase “other agents or therapies” is meant to describe additional medicinal compounds or treatments that are administered in treating a leukemia or cancer patient. Typical treatments for leukemia involve chemotherapy and/or bone marrow transplantation and/or radiation therapy. Chemotherapy in leukemia generally involves a combination of two or more anti-cancer drugs/agents. Other types of therapies include radiation therapy, which involves the use of high energy rays.

In our study, we sought to increase the cytotoxicity of thalidomide by combining it with other chemotherapeutic agents. One agent tested was interleukin-2 (IL-2) [Proleukin®, Aldesleuken for injection]. Some evidence indicates that IL-2 may be an agent which will enhance the antileukemic effect of thalidomide. This evidence is supported by studies conducted to develop an approach to prevent relapse after autologous hemopoietic cell transplantation (AHCT). Post-transplant relapse is due to minimal residual disease (MRD) in the body of the patient persisting after the conditioning regimen and/or to the presence of leukemic cells in the auto graft. After allogeneic transplant, the rate of leukemic relapse is lower for two reasons: the graft is obviously free of leukemic cells and, more importantly, because the graft-versus-leukemia (GVL) effect associated with the transfer of donor T- and natural killer (NK) cells can immunologically eradicate host leukemic cells. This knowledge has encouraged several attempts to lower leukemic relapse after AHCT by either purging the leukemic cells in the auto graft or inducing an autologous GVL effect by immunotherapy. As the results of those two approaches were not always satisfactory, other approaches which actively eradicate the residual disease from the patient's body rather than those that only eliminate the contaminating leukemic cells in the graft were done to ensure long term disease-free survival and cure. Reported immunotherapeutic approaches are diverse but most of them include the use of in vivo IL-2 with or without adoptively transferred lymphokine activated killer (LAK) cells. However, this approach is limited by the fact that the doses of IL-2 required to maintain LAK activity in vivo cause undesirable side effects.

Arsenic trioxide (Trisenox®) is currently being used to treat first relapse of acute promyelocytic leukemia (APL). The detailed mechanisms of As₂O₃ cytotoxicity are not completely known, but many preclinical studies have provided insight into the processes involved. The mechanisms include cellular differentiation, induction of apoptosis, degradation of specific APL transcripts, antiproliferation, and inhibition of angiogenesis. Many of the studies examining the activity of arsenic have used the prototype APL cell line NB4, which carries the t (15;17) translocation involving the RAR-α and PML genes. This generates a PML/RAR-α fusion protein between RAR-α, a nuclear receptor for retinoic acid and PML, a growth suppressor localized on nuclear-matrix-associated bodies. In APL studies, As₂O₃ induced a differential effect that was shown to be dose dependent: preferentially induced partial differentiation at low concentrations (0.1-0.5 mmol/l) and induced apoptosis at relatively high concentrations (0.5-2.0 mmol/l). Apoptosis in APL patients has been demonstrated to be in part secondary to down regulation of bcl-2 gene expression at protein and mRNA levels as well as through modulation of PML-RAR-α and PML. Down regulation of bcl-2 protein is independent of PML and PML/RAR-α expression. Researchers have shown that the apoptotic effect of As₂O₃ in APL is dependent in part on JNK activation (Davison et al., JNK Activation Is A Mediator of Arsenic Trioxide-Induced Apoptosis In Acute Promyelocytic Leukemia Cells, Blood, 2004; 103 (9): 3496-502. E pub 2003 Dec. 30). The antileukemic effects of all-trans retinoic acid and As₂O₃ target RAR-α and PML respectively, while both induce the degradation of PML/RAR-α fusion proteins in NB4 cells. As₂O₃ induces degradation of PML/RAR-α (as well as the wild type PML) over a wide range of concentrations (0.5-2.0 mmol/l). Moreover, As₂O₃ has been shown to induce the degradation of the PML/RAR-α fusion protein in retinoic acid resistant cells.

In related hematologic malignancies such as multiple myeloma and lymphoma, preclinical studies of As₂O₃ have demonstrated similar apoptotic effects. It was demonstrated that As₂O₃ induced G1 and/or G2M phase arrest in myeloma cells (Park et al., Arsenic Trioxide-Mediated Growth Inhibition In MC/CAR Myeloma Cells Via Cell Cycle Arrest In Association With Induction Of Cyclin Dependent Kinase Inhibitor, p21, And Apoptosis, Cancer Res. 2000; 60:3065-71). There was simultaneous induction of cyclin-dependent kinase inhibitor, p21. Researchers showed As₂O₃ induced apoptosis in resistant cell lines and fresh myeloma cells through p53-dependent cell cycle arrest and through activation of extrinsic and intrinsic caspase pathways (Liu, et al., Arsenic Trioxide—Induced Apoptosis in Myeloma Cells: p-53-Dependent G1 or G2/M cell Cycle Arrest Activation of Caspase-8 or Caspase-9, And Synergy with APO2/TRAIL, Blood, 2003; 101: 4078-87). There is also evidence of an immune mechanism with As₂O₃ in myeloma cells with elevated lymphokine activated killer cells (LAK) and other immune cells. A similar immune mechanism has not been demonstrated in As₂O₃ treated APL.

The anti-leukemic effect of As₂O₃ may in part be related to inhibition of angiogenesis by interrupting the reciprocal stimulant loop between endothelial cells releasing cytokines which stimulate leukemic cells to release growth factors such as Vascular Endothelial Growth Factor [VEGF] (through apoptosis of both cell types). Another mechanism of As₂O₃-induced apoptosis is through activation of caspases. As₂O₃ activates these proteases, which play an important role in the degradation phase of apoptosis, in NB4 cell lines. As₂O₃ can lead to membrane potential changes and increased membrane permeability with resultant degradation phase of apoptosis. Furthermore, the ability of As₂O₃ to induce apoptosis is dependent on the generation of reactive oxygen species (ROS). This suggests that the effect of As₂O₃ may be potentiated through modulation of the glutathione redox system.

The composition of thalidomide and an arsenical compound can be used in combination with another agent or therapy method, preferably another cancer treatment. The inventive composition may precede or follow the other agent treatment by intervals ranging from minutes to weeks, as shown in US Patent Application No. 2008/0090904, incorporated herein by reference.

Cancers that can be treated with the compositions taught herein include cancer of the blood cells especially acute myelogenous leukemia, bone marrow, brain, lung, liver, spleen, kidney, lymph node, small intestine, pancreas, bone, colon, stomach, breast, endometrium, prostate, testicle, ovary, central nervous system, skin, head and neck, and esophagus.

Pharmaceutical compositions can be prepared from the combination of active ingredients (thalidomide and arsenical compound) in combination with pharmaceutically acceptable carriers as set forth below.

The pharmaceutical compositions may be employed in powder or crystalline form, in liquid solution, or in suspension. The compositions are desirably administered orally; however, they may be also administered parenterally by injection.

Compositions for injection may be prepared for a desired dosage form or dose container. The injectable compositions may take such forms as suspensions, solutions or emulsions, or emulsions in oily or aqueous vehicles, and may contain various formulating agents. In injectable compositions, the carrier is typically comprised of sterile water, saline or other injectable liquid, e.g., peanut oil for intramuscular injections. Also various buffering agents, preservatives and the like can be included.

Oral formulations may take such forms as tablets, capsules, oral suspensions and oral solutions. The oral compositions may utilize carriers such as conventional formulation agents, and may include sustained release properties as well as rapid delivery forms. The dosage to be administered depends to a large extent on a variety of factors, including the condition, size and age of the subject being treated, the route and frequency of administration, and the renal and hepatic function of the subject. An ordinarily skilled physician can readily determine and prescribe the effective amount of drug required to treat, prevent, inhibit (fully or partially) or arrest the progression of the disease.

Oral/intravenous dosages of the composition may be used to treat the desired cancer, either as the composition of thalidomide and an arsenical compound, or as part of a combination therapy comprising the composition of thalidomide and an arsenical compound in combination with an additional therapy. Suitable dosage ranges for the thalidomide component based on body weight are from about 0.65 to about 12.0 mg per kg body weight per day (mg/kg/day), and more preferably from about 0.7 to 2.9 mg/kg/day.

Acceptable dosage ranges for the arsenical compound based on body weight are from about 0.10 to 0.35 mg/kg/day, more preferably from about 0.15 to 0.25 mg/kg/day.

The following examples more fully illustrate the preferred embodiments of the invention. They should in no way be construed; however, as limiting the broad scope of the invention, as described herein.

### EXAMPLES

### Materials and Methods:

The KG-1a cells, which are an early phenotype of human acute myeloid leukemia (American Type Culture Collection, Manassas, Va.), were grown in complete growth medium [Iscove's Modified Dulbeco's Medium (American Type Culture Collection, Manassas, Va.)] supplemented with 20% fetal bovine serum (Sigma-Aldrich, UK) and 1% Penicillin-Streptomycin (GIBCO Invitrogen Corporation, Carlsbad, Calif.) at 37° C. in a humidified 5% CO₂ incubator.

### Treatment of Human KG-1a Acute Myeloid Leukemia Cells:

The KG-1a cells were cultured for 48 hours in 12-well tissue culture plates, each containing complete growth medium at a concentration of 2×10⁶ cells 1 ml, and each well contained a total volume of two milliliters. Thalidomide (TOCRIS Bioscience, Ellisville, Mo.) was added at a concentration of 5 mg/L whether used alone or in combination with other chemotherapeutic agents. Interleukin-2 (Proleukin®, Aldesleuken for injection) (CHIRON Therapeutics, Emeryville, Calif.) was added at a concentration of 200 IU/ml whether used alone or in combination with thalidomide or arsenic trioxide. Arsenic trioxide (Sigma-Aldrich, Inc., St. Louis, Mo.) was added at concentrations of 2 μM and 4 μM with and without 100 μM of ascorbic acid in the first flow cytometry study, and at 4 μM in the rest of the studies either alone or combined with thalidomide and interleukin-2. A control culture containing neither thalidomide nor interleukin-2 nor arsenic trioxide was set up in conditions otherwise identical. All the control and treated cultures were set in duplicate and incubated for 48 hours at 37° C. in a humidified 5% CO₂ incubator. The incubation time was chosen to allow adequate time for apoptosis and necrosis to occur in the KG-1a cells (modified from what was published by Lu C and Hassan HT. Human Stein Cell Factor-Antibody (anti SCF) Enhances Chemotherapy Cytotoxicity in Human CD34+ Resistant Myeloid Leukemia Cells, Leukemia Research, 2006, 30(3): 296-302).

Detection of Apoptosis and Necrosis using Flow Cytometry:

The detection of apoptosis and necrosis by Annex in V-FITC assay using flow cytometric analysis was carried out as described in the Annexin V-FITC Apoptosis Detection Kit (BioVision, Inc., Mountain View, Calif.) manual. Staurosporine (Sigma-Aldrich, Inc., St. Louis, Mo.) was used as a positive control at a concentration of 10 μM and was incubated only for 24 hours with the cells. Briefly, after the KG-1a cells were incubated for 48 hours, they were harvested by centrifugation at 1,300 RPM for five minutes. Cells were resuspended in 500 μl of 1× binding buffer. Then 5 μl of Annexin V-FITC and 5 μl of propidium iodide (PI) were added to all tubes (except the negative control which contained no staining and no treatment; for the Annexin V-FITC controls only the Annexin V-FITC was added; and for the Propidium Iodide control only PI was added). All tubes were incubated at room temperature for five minutes in the dark. Then all samples were checked by the FACS Calibur Flow Cytometer (BD-Biosciences, San Jose, Calif.) and apoptotic and necrotic cells were counted according to their staining with Annexin V or PI respectively (modified from what was published in Yang, H., et al., Antileukemia Activity of the Combination of 5-aza-2′-deoxycytidine With Valproic Acid, Leukemia Research 2005; 29(7): 739-48).

### Statistical Analysis:

Results were subjected to one way ANOVA. Statistical significant differences between means was set at p<0.05.

### Example 1

### Effects of Ascorbic Acid

Flow cytometry tests were conducted to determine whether ascorbic acid (AA) enhances As₂O₃-induced cytotoxicity in the KG-1 cell line. The variant subline KG-1a of the cell line KG-1 was used as a test model. Glutathione (GSH) has been implicated as an inhibitor of As₂O₃-induced cell death either through conjugating As₂O₃ or by sequestering reactive oxygen induced by As₂O₃. Consistent with this possibility, increasing GSH levels with N-acetylcysteine attenuates As₂O₃ cytotoxicity. Decreases in GSH levels have been associated with AA metabolism, Clinically relevant doses of AA decreased GSH levels and potentiated As₂O₃-mediated cell death of all four multiple myeloma cell lines. Similar results were obtained in freshly isolated human multiple myeloma cells. Although AA is widely heralded as an antioxidant there is evidence that AA can also act as an oxidizing agent, particularly in the presence of compounds that increase the production of reactive oxygen species (ROS). The pro-oxidant effects of AA and the potentiation of cell death induced by free radicals appear to involve the production of hydrogen peroxide (H₂O₂) However, AA alone has been shown to have no effect on cell viability suggesting that AA does not produce a sufficient level of H₂O₂ to initiate oxidative damage. Rather, AA treatment increases basal levels of cellular H₂O₂. (Grad, J. M., et al., Ascorbic Acid Enhances Arsenic Trioxide-Induced Cytotoxicity in Multiple Myeloma Cell, Blood, 2001, 98(3): 805-13). Grad, et al., have shown that clinically relevant doses of AA decreases GSH levels and potentiates As₂O₃ mediated cell death of four types of multiple myeloma (MM) cell lines.

For these reasons, in the current study the possibility that ascorbic acid may increase the cytotoxicity of arsenic trioxide in the KG-1a human leukemia cell line was investigated. Two concentrations of As₂O₃ (2 μM and 4 μM) were tested in the presence or absence of AA at a concentration of 100 μM.

The results are shown in Table 1. 2 μM of As₂O₃ alone resulted in 6.88% late apoptosis in comparison to 7.12% of late apoptosis in the cells treated with As₂O₃ and AA. In addition, late apoptosis induced by 4 uM of As₂O₃ alone was 8.31% compared to 8.14% in the cells treated with As₂O₃ and AA. These findings clearly indicate that in our protocol, ascorbic acid did not enhance the cytotoxicity of arsenic trioxide. Therefore, subsequent studies were conducted without adding ascorbic acid to arsenic trioxide.

TABLE 1
Arsenic trioxide with and without ascorbic acid

| | % Live | % Early | % Late | % Necrotic |
|---|---|---|---|---|
| | Cells | Apoptosis | Apoptosis | Cells |
| As₂O₃ 2 μM | 75.94 | 14.50 | 6.88 | 2.68 |
| As₂O₃ 2 μM and | 76.03 | 15.69 | 7.12 | 1.16 |
| ascorbic acid 100 μM | | | | |
| As₂O₃ 4 μM | 69.56 | 20.87 | 8.31 | 1.26 |
| As₂O₃ 4 μM and | 69.25 | 21.84 | 8.14 | 0.77 |
| ascorbic acid 100 μM | | | | |

### Examples 2-8

### Thalidomide Alone and in Combination with IL-2 and Arsenic Trioxide

Flow cytometry tests were conducted to evaluate the efficiency of thalidomide in the management of acute myeloid leukemia (AML) and test the possibility of increasing its cytotoxicity by combining it with the other chemotherapeutic agents interleukin-2 (IL-2) and arsenic trioxide (As₂O₃).

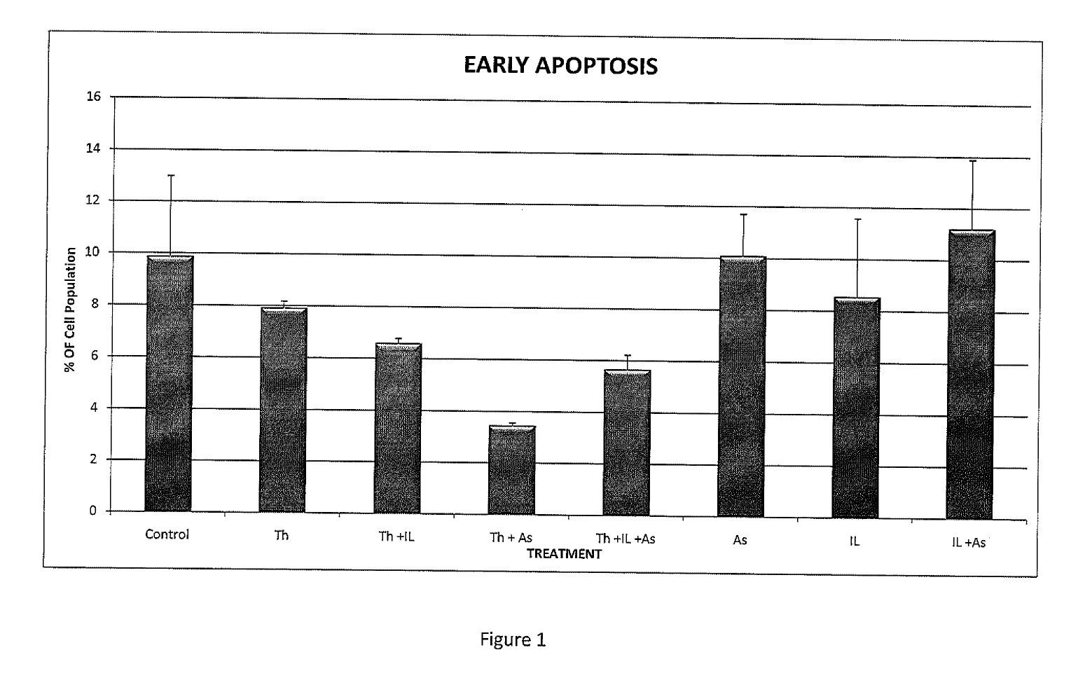

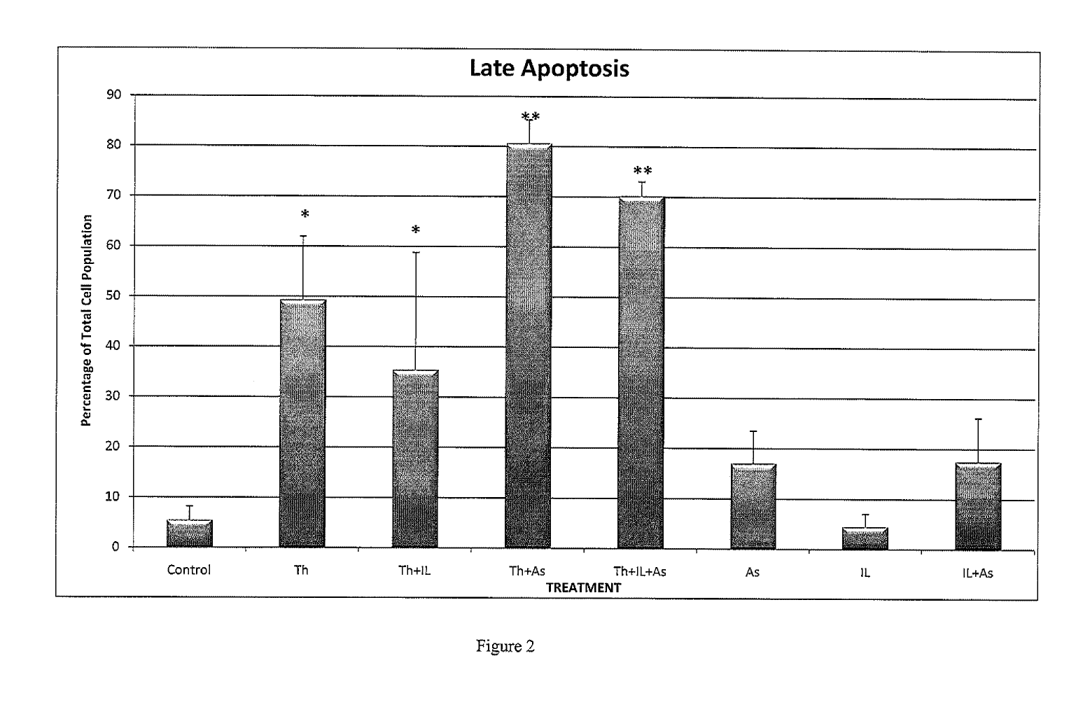

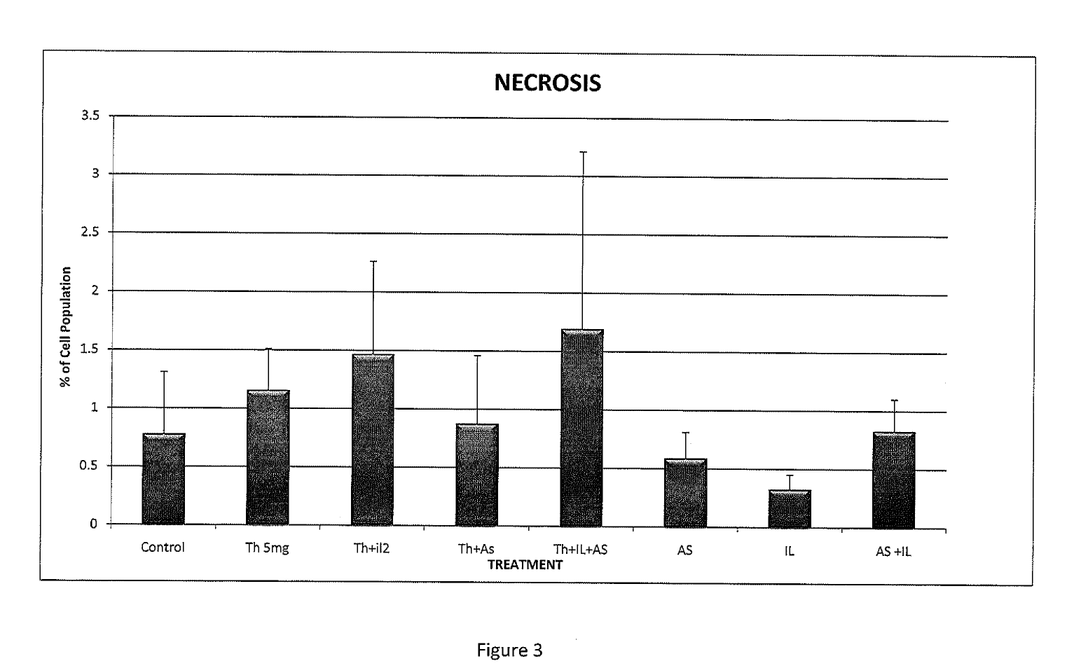

The results are shown in FIGS. 1, 2, and 3 and Table 2. Table 2 indicates the percentage of cells in each stage as measured by flow cytometry.

TABLE 2
Effect of thalidomide with other agents

| | % Early | % Late | |
|---|---|---|---|
| | apoptosis | apoptosis | % Necrosis |
| Control | 9.89 | 5.345 | 0.775 |
| Thalidomide | 7.92 | 49.385 | 1.155 |
| As₂O₃ | 10.095 | 16.965 | 0.59 |
| IL2 | 8.54 | 4.585 | 0.325 |
| Thalidomide + IL2 | 6.58 | 35.385 | 1.47 |
| Thalidomide + As₂O₃ | 3.455 | 80.6 | 0.87 |
| Thalidomide + As₂O₃ + IL2 | 5.655 | 70.135 | 1.69 |
| As₂O₃ + IL2 | 11.21 | 17.405 | 0.825 |

### Example 2

### Thalidomide Alone

Thalidomide at 5 mg/L (19 μM) resulted in 49.385% late apoptosis in comparison with 5.345% of the control. The results indicate that thalidomide exerts significant toxicity on the KG-1a cells and this cytotoxicity is mainly due to late apoptosis. This result is consistent with what was reported in Du, G. J. et al., Thalidomide Inhibits Growth of Tumors Through COX-2 Degradation Independent of Antiangiogenesis, Vascular Pharmacology, 2005, 43: 112-119, which stated that thalidomide could inhibit tumor growth in a concentration-dependent manner in MCF-7 and HL-60 cell lines and its IC₅₀s (inhibitory concentration of 50%) for them were 18.36±2.34 and 22.14±2.15 μM, respectively.

### Example 3

### Arsenic Trioxide Alone

Arsenic trioxide alone resulted in 16.97% late apoptosis in comparison to 5.345% late apoptosis in the control. The results indicate a modest cytotoxic effect.

### Example 4

### Interleukin-2 Alone

IL-2 alone exhibits a cytotoxic effect that is statistically insignificant from the control.

### Example 5

### Thalidomide Combined with Interleukin-2

The combination of IL-2 and thalidomide showed an insignificant enhancement of cytotoxicity over thalidomide alone. Several other cytokines including IL-1, IL-4, IL-7, IL-12 and granulocyte-macrophage colony-stimulating factor (GM-CSF) have been shown to be able to induce LAK activity themselves or in combination with IL-2. Rojas et al. demonstrated that immunotherapy with IL-2 plus GM-CSF after total body irradiation (TBI) results in a net improvement in survival in BALB/C mice injected with LSTRA leukemic cells. Others also reported both in vitro and in vivo generation of LAK activity by the synergistic effects of IL-2 and GM-CSF but as yet the ability of this approach to cure leukemia or to reduce the post-transplant relapse rate has not been conclusively demonstrated. We hypothesize that perhaps the combination of thalidomide and IL-2 would be more effective if a cytokine like GM-CSF was added to IL-2 (as evidenced by previous work).

### Example 6

### Thalidomide Combined with Arsenic Trioxide

When As₂O₃ was used concurrently with thalidomide it resulted in 80.6% late apoptosis in comparison to 49.39% late apoptosis of thalidomide alone. These results clearly show that the cytotoxic effect of thalidomide is significantly enhanced when combined with arsenic trioxide in the KG-1a human acute myeloid leukemia cell line. In addition, the cytotoxic effect is synergistic since the additive cytotoxicity would be 66.34, as opposed to the actual effect of 80.6% in late apoptosis.

These findings point also to the potential use of arsenic trioxide in combination with thalidomide for a more efficient management of acute myeloid leukemia with increasing the rate of complete remission for AML patients and with also the least chance of relapse. The results obtained provide promise that thalidomide effectiveness in the therapy of acute myeloid leukemia could be significantly enhanced by the concurrent use of arsenic trioxide. This will help patients who suffer from this fatal disease.

### Example 7

### Thalidomide Combined with Arsenic Trioxide and Interleukin-2

The combination of thalidomide, arsenic trioxide and interleukin-2 resulted in 70.14% of late apoptosis. These results indicate that the addition of IL-2 to the combination of thalidomide and arsenic trioxide did not enhance cytotoxicity.

### Example 8

### As₂O₃+IL2

The combination of arsenic trioxide and interleukin-2 (with no thalidomide) was not substantially different from the arsenic trioxide alone.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A pharmaceutical composition having anti-cancer activity comprising thalidomide, an arsenical compound, and a pharmaceutical carrier.

2. The pharmaceutical composition of claim 1, wherein the recommended daily dosage of the composition comprises from about 0.65 to about 12.0 mg/kg/day of thalidomide and from about 0.10 to about 0.35 mg/kg/day of an arsenical compound.

3. The pharmaceutical composition of claim 1, wherein the recommended daily dosage of the composition comprises from about 0.7 to about 2.9 mg/kg/day of thalidomide and from about 0.15 to about 0.25 mg/kg/day of an arsenical compound.

4. The pharmaceutical composition of claim 1, wherein the composition has anti-cancer activity in cancers selected from the group consisting of cancer of the blood cells especially acute myelogenous leukemia, bone marrow, brain, lung, liver, spleen, kidney, lymph node, small intestine, pancreas, bone, colon, stomach, breast, endometrium, prostate, testicle, ovary, central nervous system, skin, head and neck, or esophagus.

5. The pharmaceutical composition of claim 1, wherein the arsenical compound is arsenic trioxide (As₂O₃).

6. A pharmaceutical composition adapted for oral administration, comprising thalidomide, an arsenical compound, and a pharmaceutical carrier.

7. The pharmaceutical composition of claim 6, wherein the arsenical compound is arsenic trioxide (As₂O₃).

8. A method of treating a patient with cancer comprising administering to the individual a composition comprising therapeutically effective amounts of thalidomide and an arsenical compound.

9. The method of claim 8, wherein said therapeutically effective amounts are from about 0.65 to about 12.0 mg/kg/day of thalidomide and from about 0.10 to about 0.35 mg/kg/day of an arsenical compound.

10. The method of claim 8, wherein said therapeutically effective amounts are from about 0.7 to about 2.9 mg/kg/day of thalidomide and from about 0.15 to about 0.25 mg/kg/day of an arsenical compound.

11. The method of claim 8, wherein said cancer comprises a solid tumor.

12. The method of claim 11, wherein said cancer is cancer of the blood cells, bone marrow, brain, lung, liver, spleen, kidney, lymph node, small intestine, pancreas, bone, colon, stomach, breast, endometrium, prostate, testicle, ovary, central nervous system, skin, head and neck, or esophagus.

13. The method of claim 8, wherein said cancer is a hematological cancer.

14. The method of claim 13, wherein said cancer is leukemia, lymphoma, multiple myeloma, myelodysplasia, myeloproliferative disease, or refractory anemia.

15. The method of claim 14, wherein said cancer is acute myeloid leukemia.

16. The method of claim 8, wherein the composition is administered daily.

17. The method of claim 8, wherein the composition is administered orally.

18. The method of claim 8, wherein a dose of the composition is administered with one or more other agents or therapies.
